(19) United States
(12) Patent Application Publication
(10) Pub. No.: US 2014/0046170 A1
(43) Pub. Date: Feb. 13, 2014

(54) BRAIN VOLUMETRIC MEASURING METHOD AND SYSTEM USING THE SAME

(71) Applicants: Chia-Wei Sun, New Taipei City (TW); Ching-Cheng Chuang, Taichung City (TW); Yao-Sheng Hsieh, New Taipei City (TW)

(72) Inventors: Chia-Wei Sun, New Taipei City (TW); Ching-Cheng Chuang, Taichung City (TW); Yao-Sheng Hsieh, New Taipei City (TW)

(21) Appl. No.: 13/568,614

(22) Filed: Aug. 7, 2012

(51) Int. Cl. A61B 5/107; A61B 5/055; A61B 6/00

(52) U.S. Cl. 600/411; 600/476

(57) ABSTRACT

The present invention discloses a brain volumetric measuring method for measuring brain volumetric changes of a subject. The method at least comprises the following steps. First, a light source is provide and emitted into the head of the subject through a light source emitting position. And then, a first optical signal is obtained by receiving numerous scattered photons from the head of the patient through several second positions of. A second optical signal will be obtained by processing the first optical signal. The present invention also discloses a brain volumetric measuring system for performing the abovementioned method.

### CROSS-REFERENCE TO RELATED APPLICATIONS

### Field of the Invention

This invention relates to a brain volumetric measuring method, especially relates to a brain volumetric measuring method performed by using a near-infrared diffuse optical imaging technique to analysis and observe the changes of brain volume.

### BACKGROUND OF THE INVENTION

Brain atrophy is an irreversible brain disease that causes problems with cognitive and memory functions in many diseases, such as mild cognitive impairment, Alzheimer disease (AD), multiple sclerosis, schizophrenia, alcoholism, dementia, etc. The neuropathological process of brain atrophy involves progressive biochemical and structural changes that begin at the cellular and synaptic level, and ultimately culminate in neuronal death, loss of nerve cells, white matter (WM) and gray matter (GM) degeneration. The loss of neurons and synapses in the cerebral cortex and subcortical regions results in gross atrophy of the affected regions, including degeneration in the hippocampus, temporal lobe, parietal lobe, and frontal cortex.

Basically, there are lots of reasons causing brain atrophy, such as traumatic brain injury, cerebral embolism, meningitis, cerebral vascular malformations, brain tumor, epilepsy, long-term drinking, malnutrition, hypoparathyroidism, cerebral dysplasia, abuse of sedatives, gas poisoning, alcoholism, chemical poisoning etc. Three clinical stages of brain atrophy are early stage, middle stage and late stage. The most common symptoms of early stage are hypomnesis and decline in thinking ability. The symptoms of middle stage are obviously deterioration of memory especially for recent events, at the same time, forgotten of remote memory and beginning to have distinct cognitive dysfunction. At last, the patients in the late stage are dementia, walk with difficulty and needed support, bedridden or stay in the seat, disorientation. Therefore, as abovementioned, brain atrophy is not only an irreversible but progressive disease, which makes the brain volumetric changes becoming an important clinical indicator.

Neuroimaging and related analytical research is an accurate, reproducible and quantitative measure, which is getting more attention and widely used in assessing brain volumetric changes. Current neuroimaging modalities, such as magnetic resonance imaging (MRI), computed tomography (CT), and positron emission tomography (PET), could be used to identify brain regional atrophy characteristics and predict cognitive decline of patients with mild cognitive impairment as well as the structural changes of brain atrophy.

Brain atrophy mostly happens on elderly. However, in the case of MRI, the limitation of a large size of instruments that cannot move around arbitrary leads to the incapability of providing diagnosis with patient-oriented measurement, producing real-time images and conducting long-term monitoring of the pathological changes. Moreover, this technique could not be used to diagnose patients with claustrophobic.

Recently, the near-infrared diffuse optical imaging (DOI) technique could be used to detect brain-related functional neural activity by using light sources of several wavelengths in near-infrared range to perform calculation with different absorption coefficients of oxyhemoglobin and deoxyhemoglobin and get the oxygen concentrations varies with the brain activities for real-time measurement of oxygen changes in the brain tissue. However, there is no current study utilizing optical imaging technique to measure the structural volumetric changes of brain atrophy.

### SUMMARY OF THE INVENTION

Accordingly, the present invention provides a brain volumetric measuring method used to real-time monitoring the brain volumetric changes of a subject. The brain atrophy will cause the brain structural volumetric changes. The most obvious atrophy phenomenon is decreasing of greymatter and whitematter, which results in the volumetric increase of cerebrospinal fluid. Basically, the low scattering and low absorption optical characteristic of cerebrospinal fluid results in great light-guiding efficiency of light channel effect in brain. Coordinate with the asymmetry and expend of light channel of brain atrophy, one could use the abovementioned near-infrared light source to perform imaging and quantitative analysis by measuring the light distribution pattern of the light signal in brain affected by the brain structural changes.

The abovementioned brain volumetric measuring method at least comprises the following steps: First, a light source is provide and emitted into the head of the subject through a light source emitting position. And then, a first optical signal is obtained by receiving numerous scattered photons from the head of the patient through several light source receiving positions. A second optical signal will be obtained by processing the first optical signal.

Preferably, the structural differences of the brain affect the distribution of the light when the light of the light source passes through the head of the subject.

Preferably, the light source is a single-band near-infrared illumination or a multi-band near-infrared illumination. Furthermore, the light source emitting position and the light source receiving positions are placed along a transverse cross section, a sagittal cross section and a coronal cross section of the head of the subject, and the light source emitting position and the light source receiving positions are not overlapped. When the light source emitting position and the light source receiving positions are placed along the transverse and sagittal cross sections, the light source emitting position is put on the middle of the forehead and 6 cm deep from the top of the head of the subject, and the distance between the light source emitting position and the light source receiving positions are from 1 to 5 cm, separately. When the light source emitting position and the light source receiving positions are placed along the coronal cross section, the light source emitting position is put on the top of the middle head of the subject, and the distance between the light source emitting position and the light source receiving positions are from 1 to 5 cm, separately.

Preferably, the brain volumetric measuring method further comprises the following steps: First, a database is provided with a plurality of pathological classifications. Furthermore, each pathological classification contains a plurality of brain structural atrophy degrees. Accordingly, the abovementioned brain volumetric measuring method further comprises the following steps: a step of comparing the second optical signal with the database is performed at first. And then, a classify result of the brain structural atrophy degree is received.

Preferably, the step of comparing the second optical signal and the database further comprising the following steps: a step of classifying the second optical signal into one of the pathological classifications is performed at first. And then, it will be determined whether the second optical signal matches a critical value of the one of the pathological classification. If the second optical signal matches the critical value, the subject possesses a brain structural abnormality. A step of comparing the brain structural abnormality with the brain structural atrophy degrees is followed, and a result is then obtained and displayed. In the preferred embodiment, the result corresponds to one of the brain structural atrophy degrees.

Preferably, the step of processing the first optical signal to get the second optical signal is performed by using a m×n multi-point brain volumetric measurement algorithm.

Preferably, the brain volumetric measuring method further comprises the following step: a step of combining the first optical signal with a MRI image of the head to build a model of brain tissue is performed by using a Monte Carlo simulation.

Another purpose of the present invention is to provide a brain volumetric measuring system for performing the abovementioned brain volumetric measuring method. The system at lease comprises an optical device and an assessment device. The optical device comprises an optical probe for emitting a light and a plurality of detectors for receiving numerous scattered photons. And further, the optical probe is placed at a light source emitting position to let the light enter the head of the subject, and the detectors are placed at a light source receiving positions to receive the scattered photons to get a first optical signal. The assessment device is used for processing the first optical signal to get a second optical signal.

Preferably, the light source receiving positions are not overlapped and the distances existed between the optical probe and each one of the detector are different.

Preferably, the optical device further comprises a signal processing circuit for amplifying and filtering the first optical signal.

Preferably, the brain volumetric measuring system further comprises a transmission device, and the transmission device disposes between the optical device and the assessment device for capturing the first optical signal and transmitting the first optical signal into the assessment device.

Preferably, the transmission device is a data acquisition card, a digital-to-analog converter, an analog-to-digital converter or a single chip.

Preferably, the brain volumetric measuring system further comprises a light source for producing the light and the light source is a single-band near-infrared illumination or a multi-band near-infrared illumination.

Preferably, the optical probe is a m×n optical array probe and the first optical signal is a brain optical array signal.

Preferably the assessment device processes the first optical signal by using a m×n multi-point brain volumetric measuring algorithm so that the second optical signal is a brain volumetric optical signal, and the assessment device is further used for comparing the second optical signal with a plurality of brain structural atrophy degrees within different pathological classifications of a database to obtain a result.

Preferably, the assessment device is further used for building a brain tissue model of the subject.

Preferably, the assessment device further comprises a display unit for real-time displaying the second optical signal, the result or the brain tissue model.

The features and advantages of the present invention will be understood and illustrated in the following specification and FIGS. 1˜8.

### BRIEF DESCRIPTION OF THE DRAWINGS

FIG. 1 is a diagram showing modeling process of 3-D human brain from in vivo MRI T1 images wherein FIG. 1a shows 2-D anatomical MIR slice. FIG. 1b shows Segmentation of scalp and skull, FIG. 1c shows Segmentation of CSF layer, FIG. 1d shows Segmentation of GM, FIG. 1e shows Segmentation of WM, FIG. 1f shows 2-D optical brain model in five-layer brain structure, FIG. 1g shows 3-DMRI T1 image and FIG. 1h shows 3-D optical brain model;

FIG. 2 is a diagram showing the flow chart of the brain modeling process by means of the Monte Carlo algorithm with MRI data;

FIG. 3 is a diagram showing the frame diagram of brain volumetric measuring system according to the present invention;

FIG. 4 is a diagram showing the flow chart of the brain volumetric measuring method according to the present invention;

FIGS. 5A and 5B is a diagram showing the arrangement of source-detector separations according to the present invention;

FIG. 6 is a diagram showing the transverse view of the DOI and analyzed result of three subjects according to the present invention;

FIG. 7 is a diagram showing the sagittal view of the DOI and analyzed result of three subjects according to the present invention; and

FIG. 8 is a diagram showing the coronal view of the DOI and analyzed result of three subjects according to the present invention.

### DETAILED DESCRIPTION OF THE INVENTION

Recently, near-infrared DOI technique has drawn more and more attention due to several advantages, such as noninvasive, less expensive, non-ionizing radiation, long-time monitoring, not restricted by space and easy operation. Therefore, in consideration of the conventional problem, the present invention provides an imaging and quantitative assessment of the degree of brain atrophy by utilizing a near-infrared diffuse optical imaging technique to measure the distribution of light in brain affected by brain atrophy-induced structural volumetric change. However, near-infrared is limited by strong scattering in biological tissue, the energy of light attenuates severely with the distance of light passing through biological tissue, and this phenomenon seriously affects the penetration depth of light in tissue. Although the penetration depth of near-infrared is only 3 cm, it is enough for this invention to measure the activity of cerebral cortex and the structural volumetric changes caused by brain atrophy, especially in interhemispheric fissure of prefrontal cortex. Because the expanded CSF volume offers light-guiding channels, the system disclosed in the present invention could be a great tool to detect brain volumetric changes for patient-oriented neurodegenerative diseases diagnosis.

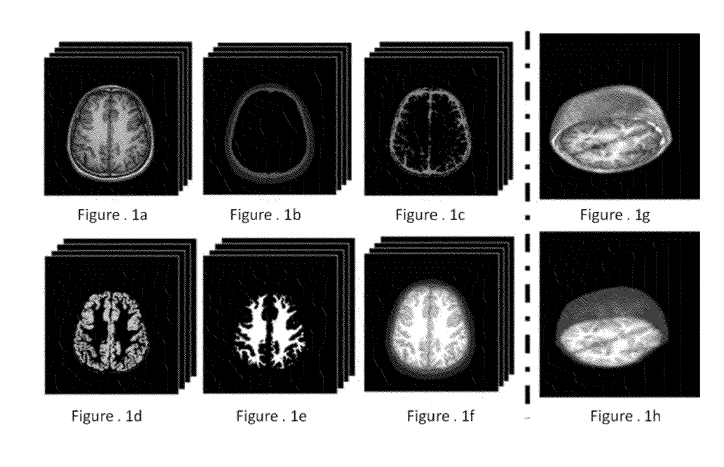

Please refer to FIGS. 1a˜1h, FIGS. 1a˜1h are diagrams showing modeling process of 3-D human brain from in vivo MRI T1 images wherein FIG. 1a shows 2-D anatomical MIR slice, FIG. 1b shows Segmentation of scalp and skull, FIG. 1c shows Segmentation of CSF layer, FIG. 1d shows Segmentation of GM, FIG. 1e shows Segmentation of WM, FIG. 1f shows 2-D optical brain model in five-layer brain structure, FIG. 1g shows 3-DMRI T1 image and FIG. 1h shows 3-D optical brain model. First, the clinical MRI T1 scan offers 92 axial slices as in FIG. 1(a). The images were segmented into five layers of brain tissue as scalp, skull, CSF, GM, and WM. The 3-D brain image contains 256×256×92 voxels and each voxel size is 1×1×1 mm³ that corresponds to the resolution of 3-D MRI image. First, the scalp and skull layers were segmented by means of edge detection and region growing as shown in FIG. 1b. Then, the probabilistic framework was applied to classify CSF, GM, and WM layers with unified segmentation, which was performed by fitting a mixture of Gaussians (MOG) model with prior information of deformable tissue probability maps as shown in FIGS. 1c, 1d, and 1e, respectively. FIG. 1f demonstrates a 2-D five-layer brain structure after segmentation processes. Finally, FIG. 1g and FIG. 1h shows the 3-D MRI image and the corresponding optical brain model that was performed in Monte Carlo simulation. In the simulations, an 800-nm wavelength light source was applied for illumination of healthy, aged, and typical AD brains. The reduced scattering coefficient μ^{L}·s, absorption coefficient μₐ, scatters' radius, refractive indices of background, and scatters of brain tissues are presented in table 1 listed as the following Table 1.

TABLE 1

| | | | Refractive | Refractive index | Radius of | |
|---|---|---|---|---|---|---|
| | μₛ′ | μₐ | index of | of scattering | scattering | Anisotropy |
| Brain tissues | (cm⁻¹) | (cm⁻¹) | background | particles | particles(μm) | factor(g) |
| Scalp | 19 | 0.18 | 1.4 | 1.565 | 10 | 0.92 |
| Skull | 16 | 0.16 | 1.4 | 1.565 | 10 | 0.92 |
| CSF | 2.4 | 0.04 | 1.331 | 1.565 | 10 | 0.92 |
| Gray matter | 22 | 0.36 | 1.36 | 1.565 | 10 | 0.92 |
| White matter | 91 | 0.14 | 1.38 | 1.565 | 10 | 0.92 |
| Air | — | — | 1 | — | — | — |

As mentioned earlier, the volume of CSF was increased with the atrophy of GM and WM. The penetrated photons could be guided along the CSF layer because of its low scattering property. This phenomenon can be used for structural characterization of brain with NIRS/DOI measurement.

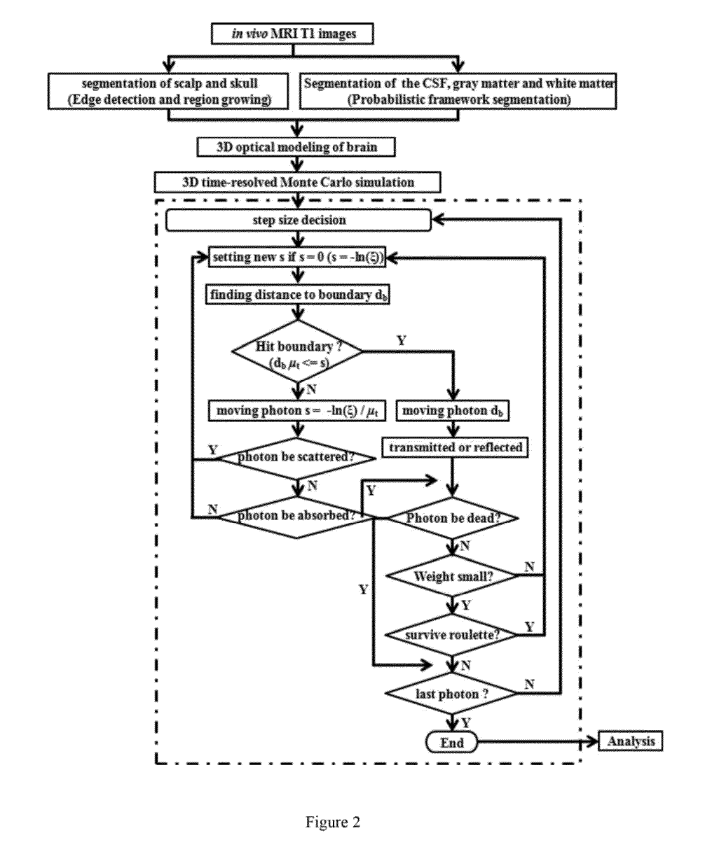

Monte Carlo modeling is a method that can be used to simulate photon interaction in turbid tissue. The Monte Carlo algorithm which inventor used is represented as the dashed line area in FIG. 2. In this practical example, the point source was used, which means that all the photons start to emit at the same direction. If the photon propagates through a voxel boundary, the step size would be modified as s=−ln(ξ)/μt, where ξ is a sampled value of a uniform random variable within the interval [0,1] and μt is an extinction coefficient. If the refractive indices are different between two adjacent voxels, the transmission or reflection then occurs alternatively. In addition, Snell's law and Fresnel reflection formulas were applied at each boundary. A scattering event, a new step size, deflection angle, and azimuthal angle were calculated for each photon. Generally, the behavior of photon migration in brain can be decided by two steps: 1) the mean free path of a scattering/absorption event and 2) the probability density function of scattering angle. The absorption and scattering properties of a sphere can be described by the Mie theory that has been available in previous studies. All of the photons are traced and recorded for photon migration analysis and imaging.

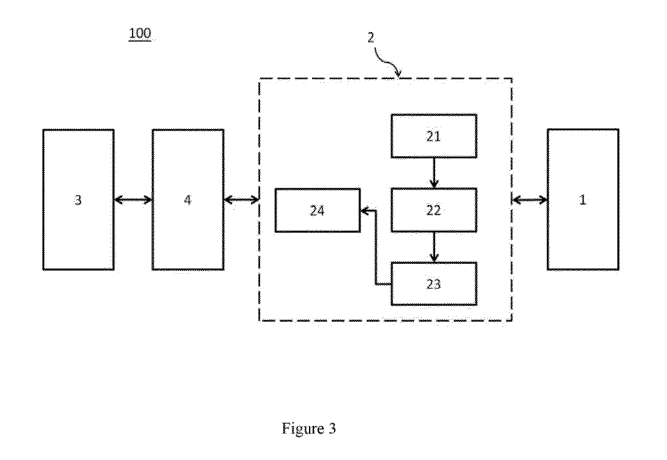

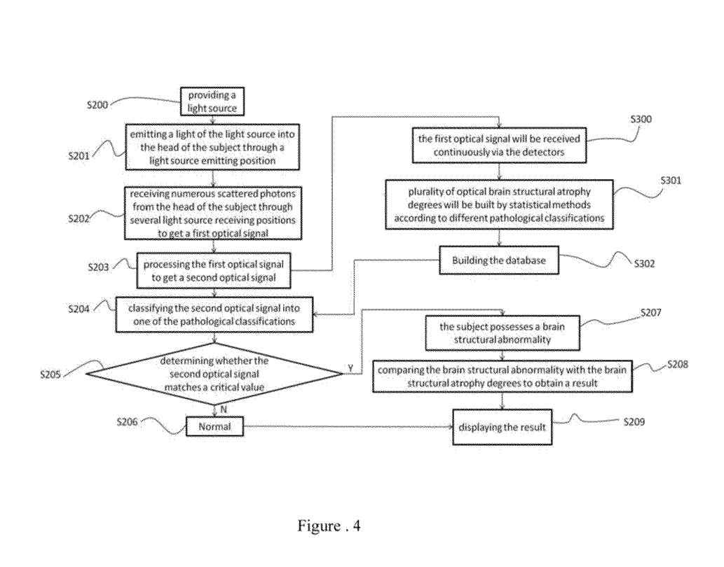

After the description of the abovementioned principle applied in the present invention, please refer to FIG. 3 and FIG. 4. FIG. 3 is a diagram showing the frame diagram of brain volumetric measuring system according to the present invention, and FIG. 4 is a diagram showing the flow chart of the brain volumetric measuring method according to the present invention. As shown in FIG. 3, the present invention provides a brain volumetric measuring method and the applying measuring system for real-time measuring the brain structural volumetric changes of a subject 1. The system 100 at least comprises an optical device 2 and an assessment device 3. The optical device 2 includes a light source 21, an optical probe 22 and a plurality of detectors 23 (in order to simplify the diagram, only one detector 23 is shown in FIG. 3). Preferably, the light source 21 is a single-band near-infrared illumination or a multi-band near-infrared illumination. Although it is not shown in diagram, the light source 21 can comprise any component which could emit near-infrared luminescence, such as laser and LED. Preferably, the optical probe 22 can be only a single m×n array probe or represents multiple m×n array probes. And further, the optical probe can be a full optical fiber probe or a non optical fiber probe, the present invention is not limited thereto. Any electronic component, including semiconductor laser, which can emit and conduct photons can be also included therein.

Preferably, the detectors 23 could be any optical signal-receiving electronic device, such as light detector and light sensor.

Preferably, the optical device 2 comprises a signal processing circuit 24 for further amplifying and filtering the signal received from detectors 23.

As to the assessment device 3, it could be a program controllable computer or single chip micro-processor device, but the present invention is not limited thereto. Besides, the present system 100 further comprises at least one transmission device 4 disposed between the optical device 2 and the assessment device 3 for transmitting the signal from the assessment device 3 to drive the optical device 2 or capturing the signal from the optical device 2 to process in the assessment device 3. Preferably, the transmission device 4 could be a data acquisition card, a digital-to-analog converter, an analog-to-digital converter or a single chip, but the present invention is not limited thereto.

The following is a detail description of the frame diagram in FIG. 4 about the brain volumetric measuring method provided by the present invention. First, a light source is offered as abovementioned S200. The light source herein means the light source 21 of the abovementioned optical device 2. Then, the optical probe 22 is placed at a light source emitting position of the head of the subject 1 so that a light emitted from the light source 21 will enter into the head of the subject 1 through the light source emitting position S201. The detectors 23 are arranged on a plurality of light source receiving positions of the head of the subject 1 for receiving numerous scattered photons to get a first optical signal S202. Finally, the assessment device 3 processes the first optical signal to get a second optical signal S203 after transmitting the first optical signal to the assessment device 3 by the transmission device 4. Preferably, the first optical signal is a brain optical array signal because the optical probe 22 is am×n optical array probe. In addition, the assessment device 3 processes the first optical signal by utilizes a m×n multi-point brain volumetric measuring algorithm, and the second optical signal is a brain volumetric optical signal.

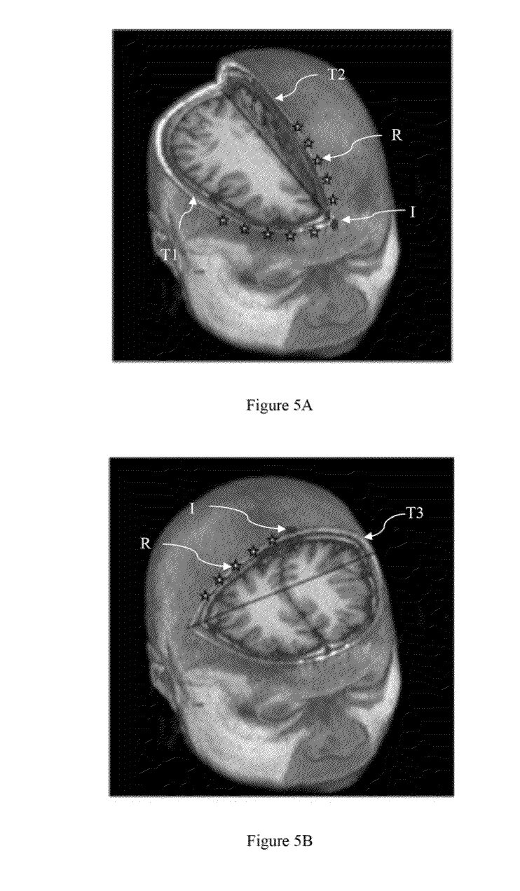

Please refer to FIG. 5A and FIG. 5B. FIG. 5A and FIG. 5B is a diagram showing the arrangement of source-detector separations. Briefly, the present invention utilizes different distances between light source and detectors to create images by capturing the attenuate light signal. The light source emits the light into the head of the subject through the optical probe, and the light source emitting position of the optical probe and the light source receiving positions of detectors are arranged along a transverse cross-section T1, a sagittal cross-section T2, or a coronal cross-section T3.

As shown in FIG. 5A, the circular marker 1 represent the position of the optical probe (that is, the position of emitting the light into the head or the light source emitting position). The multiple star markers represent the light source receiving positions and the light source receiving positions are not overlapped. Preferably, when the light source emitting position (circular marker 1) and the light source receiving positions (star marker R) are separated and placed along the transverse cross-section T1 or the sagittal cross-section T2, the light source emitting position is put on the middle of the forehead and 6 cm deep from the top of the head of the subject, and the distance between the light source emitting position and the light source receiving positions are from 1 to 5 cm, separately. That is, the light source and the detectors possess the intervals, and those intervals different from each other as the abovementioned. In another preferred embodiment as shown in FIG. 5B, when the light source emitting position and the light source receiving positions are placed along the coronal cross section, the light source emitting position is put on the top of the middle head of the subject, and the distance between the light source emitting position and the light source receiving positions are from 1 to 5 cm, separately.

Please refer to FIG. 4 again. The brain volumetric measuring method disclosed in the present invention further comprises the following steps: the first optical signal will be received continuously via the detectors and the first optical signals will be transmitted to the assessment device to be processed and get a plurality of the second optical signals. The assessment device can perform massive information search through current clinical diagnosis and brain structural medical imaging (such as MRI and CT), and compare the continuously arriving second optical signal with the plurality of different pathological classifications. That is, the second optical signal in the present invention can perform different brain volumetric classifications in various pathologies S300.

After step S300, plurality of optical brain structural atrophy degrees will be built by statistical methods according to different pathological classifications. S301. That is, all kinds of diseases can be divided into plurality of pathological classifications according to variety of brain volume, and each pathological classification can be separated into numerous degrees of symptoms, such as degree of atrophy. Finally, the abovementioned pathological classifications and the comprising plurality of brain structural atrophy degrees will be organized into a database S302.

Accordingly, the brain volumetric measuring method disclosed in the present invention comprises the following steps: first, the second optical signal will be classified into one of the pathological classifications in database S204. Then, the second optical signal will be decided whether it matches one of the critical value among those information in the pathological classification S205. If not, the subject will be evaluated to be normal S206. On the other hand, if it does, the subject will be evaluated to possess a brain structural abnormality S207.

Afterwards, the step of comparing with the abovementioned database and verifying which of the brain structural atrophy degree within the pathological classification matches the situation of the subject S208 is performed. And then, a result will be obtained. The abovementioned result will match one of the brain structural atrophy degrees. Finally, the abovementioned result will be displayed S209. Therefore, although it is not shown, the assessment device can further contain a screen to display the abovementioned result. However, the present invention does not limited thereto.

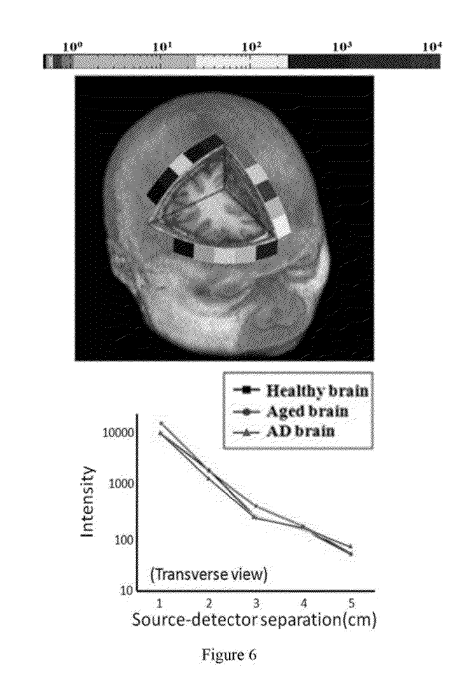

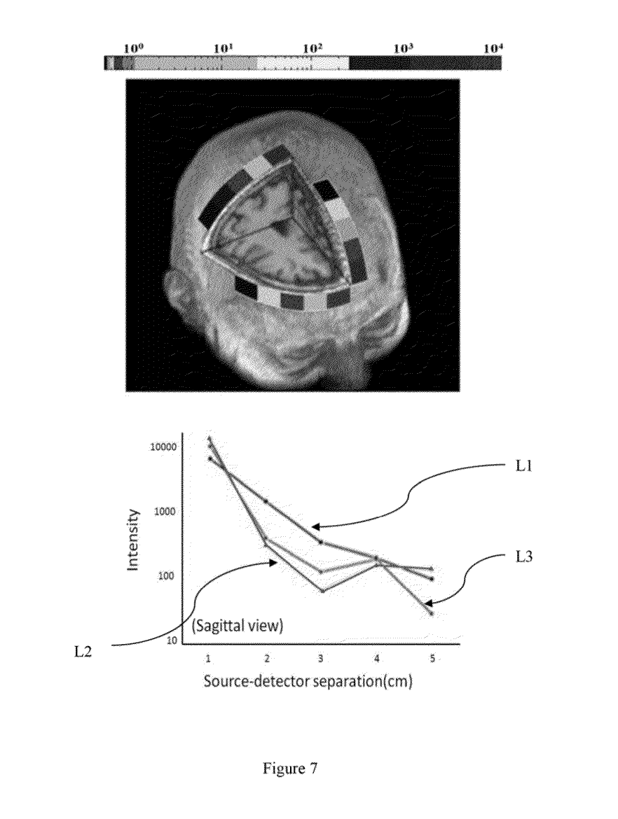

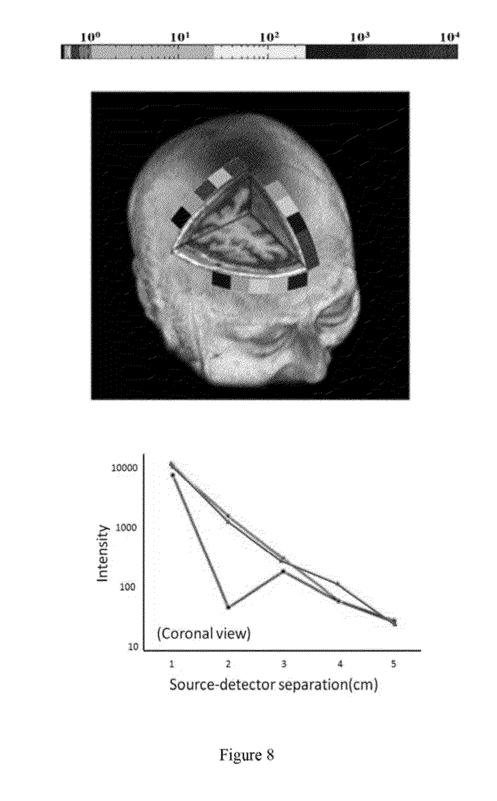

In order to prove that the present invention can effectively measure the brain structural volumetric changes, a healthy, aged, and AD) subjects were tested and get the following results. Please refer to FIG. 6 to FIG. 8. FIG. 6 is a diagram showing the transverse view of the DOI and analyzed result of three subjects according to the present invention, FIG. 7 is a diagram showing the sagittal view of the DOI and analyzed result of three subjects according to the present invention, and FIG. 8 is a diagram showing the coronal view of the DOI and analyzed result of three subjects according to the present invention. In principle, the brain tissue model of the subject in FIG. 6 to FIG. 8 could be built by the abovementioned Monte Carlo simulation combined with the first optical signal and a MRI image of the head of the subject. As shown in FIG. 6 to FIG. 8, it clearly shows that the intensity of light signal is strongly affects by the volumetric changes of greymatter, whitematter and cerebrospinal fluid caused by brain atrophy.

As shown in FIG. 6 to FIG. 8, the X axis represents that the interval between the light source and the detectors are 1 to 5 cm and the Y axis represents that the intensity of light received by the detectors. L1 is the result of the healthy subject, L2 is the result of the AD subject, and L3 is the result of the aged subject. The trends of structural changes caused by brain atrophy affecting the intensity of light could be distinguished from the data analysis of the abovementioned three cross sections.

Please refer to FIG. 6, the result of the transverse cross-section shows that there is no obvious difference of brain volumetric changes between those three subjects.

Please refer to FIG. 7, the result of the sagittal cross-section shows that the light signal of healthy subject attenuates steady with the distance between the light source and the detectors. Furthermore, the intensity of light changes severely due to the asymmetry of brain structural atrophy of AD and aged subjects.

Please refer to FIG. 8, the result of the coronal cross-section shows that in the case of AD and aged subjects, the degradation and atrophy of brain structure caused volumetric decreasing of greymatter and whitematter and increasing of cerebrospinal fluid. Therefore, steady signal attenuation occurred on AD subject due to the light-guiding effect of cerebrospinal fluid. On the other hand, multiple scattering and absorption of light signal occurred in the healthy subject due to larger volume of greymatter and whitematter that blocked the light transmission path results in a severe fluctuation of the decreasing of light intensity.

The structural differences of brain atrophy could be understood through display of images and the data classification could be accomplished by analysis of the wave shape of light signal. This could be a reference index for clinical paramedics to diagnose the degree of brain atrophy and even further understand the effectiveness of the treatment.

In summary, the present invention utilizing an optical technique to measure the brain volumetric changes. There is no any research or product measuring the brain volumetric changes through optical technique and the doctor could use hand-held probe to real-time diagnose the patient on outpatient treatment because this optical technique does not limit by space and time. Besides, this optical technique could be made into a portable system which could provide a long-term data monitoring for home-care patients helping doctors to perform long-term tracking and diagnosis, and long-term evaluation of the patient's treatment.

Moreover, the technology transfer threshold and production cost of optical device is relatively low, it would be easy for massive production. Additionally, the whole world is going into an aging society and it will provide a huge marketing orientation for home-care or long-term disease tracking. Therefore, the abovementioned optical diagnostic device not only offers a convenience for doctor, a real-time data measurement to help diagnosis or overall arrangement of home-care marketing, which are advantages MRI or CT could never achieved.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A brain volumetric measuring method for measuring brain volumetric changes of a subject, comprising the following steps:
providing a light source;
emitting a light of the light source into the head of the subject through a light source emitting position;
receiving numerous scattered photons from the head of the subject through several light source receiving positions to get a first optical signal; and
processing the first optical signal to get a second optical signal.

2. The brain volumetric measuring method according to claim 1, wherein when the light passes through the head of the subject, the differences of the brain structural affect the distribution of the light.

3. The brain volumetric measuring method according to claim 1, wherein the light source is a single-band near-infrared illumination or a multi-band near-infrared illumination.

4. The brain volumetric measuring method according to claim 1, wherein the light source emitting position and the light source receiving positions are placed along a transverse cross section, a sagittal cross section and a coronal cross section of the head of the subject, and the light source emitting position and the light source receiving positions are not overlapped.

5. The brain volumetric measuring method according to claim 4, wherein when the light source emitting position and the light source receiving positions are placed along the transverse and sagittal cross sections, the light source emitting position is on the middle of the forehead and 6 cm deep from the top of the head of the subject, and the distance between the light source emitting position and the light source receiving positions are from 1 to 5 cm, separately.

6. The brain volumetric measuring method according to claim 4, wherein when the light source emitting position and the light source receiving positions are placed along the coronal cross section, the light source emitting position is on the top of the middle head of the subject, and the distance between the light source emitting position and the light source receiving positions are from 1 to 5 cm, separately.

7. The brain volumetric measuring method according to claim 1, further comprising the following steps:
providing a database with a plurality of pathological classifications wherein each of the pathological classifications includes a plurality of brain structural atrophy degrees;
comparing the second optical signal with the database; and
receiving a classify result of the brain structural atrophy degree.

8. The brain volumetric measuring method according to claim 7, wherein the step of comparing the second optical signal and the database further comprising the following steps:
classifying the second optical signal into one of the pathological classifications;
determining whether the second optical signal matches a critical value of the one of the pathological classification, and the subject possesses a brain structural abnormality if the second optical signal matches the critical value;
comparing the brain structural abnormality with the brain structural atrophy degrees to obtain a result, wherein the result corresponds to one of the brain structural atrophy degrees; and
displaying the result.

9. The brain volumetric measuring method according to claim 1, wherein the step of processing the first optical signal to get the second optical signal is performed by using a m×n multi-point brain volumetric measurement algorithm.

10. The brain volumetric measuring method according to claim 1, further comprising the following step:
combining the first optical signal with a MRI image of the head to build a model of brain tissue by using a Monte Carlo simulation.

11. The brain volumetric measuring system for measuring brain volumetric changes of a subject, comprising:
an optical device, comprising:
an optical probe emitting a light;
a plurality of detectors receiving numerous scattered photons,
wherein the optical probe is placed at a light source emitting position to let the light enter the head of the subject, and the detectors are placed at a light source receiving positions to receive the scattered photons to get a first optical signal; and
an assessment device processing the first optical signal to get a second optical signal.

12. The brain volumetric measuring system according to claim 11, wherein the light source receiving positions are not overlapped of each other and the distances between the optical probe and each detector are different.

13. The brain volumetric measuring system according to claim 11, wherein the optical device further comprises a signal processing circuit for amplifying and filtering the first optical signal.

14. The brain volumetric measuring system according to claim 11, further comprising:
at least a transmission device disposing between the optical device and the assessment device for capturing the first optical signal and transmitting to the assessment device.

15. The brain volumetric measuring system according to claim 14, wherein the transmission device is a data acquisition card, a digital-to-analog converter, an analog-to-digital converter or a single chip.

16. The brain volumetric measuring system according to claim 11, further comprising a light source for producing the light and the light source is a single-band near-infrared illumination or a multi-band near-infrared illumination.

17. The brain volumetric measuring system according to claim 11, wherein the optical probe is a m×n optical array probe and the first optical signal is a brain optical array signal.

18. The brain volumetric measuring system according to claim 17, wherein the assessment device processes the first optical signal by using a m×n multi-point brain volumetric measuring algorithm so that the second optical signal is a brain volumetric optical signal, and the assessment device is further used for comparing the second optical signal with a plurality of brain structural atrophy degrees within different pathological classifications of a database to obtain a result.

19. The brain volumetric measuring system according to claim 18, wherein the assessment device is further used for building a brain tissue model of the subject.

20. The brain volumetric measuring system according to claim 19, wherein the assessment device further comprises a display unit for real-time displaying the second optical signal, the result or the brain tissue model.

21. The brain volumetric measuring system according to claim 11, wherein the assessment device is a program-controllable computer or a single-chip micro-processing device.
